(19) United States
(12) Patent Application Publication
(10) Pub. No.: US 2023/0001156 A1
(43) Pub. Date: Jan. 5, 2023

(54) Vascular Access Device with Non-Contact Guidewire Advancement

(71) Applicant: Becton, Dickinson and Company, Franklin Lakes, NJ (US)

(72) Inventors: Megan Scherich, Salt Lake City, UT (US); Jonathan Karl Burkholz, Salt Lake City, UT (US)

(21) Appl. No.: 17/851,666

(22) Filed: Jun. 28, 2022

Related U.S. Application Data
(60) Provisional application No. 63/218,143, filed on Jul. 2, 2021.

(51) Int. Cl. A61M 25/06; A61M 25/00; A61M 25/09; A61M 25/01

(52) U.S. Cl. CPC A61M 25/0606; A61M 25/0097; A61M 25/09; A61M 25/0113

(57) ABSTRACT

A vascular access device may include an advancement element and a housing. The housing may include a lumen extending therethrough, which may be used for blood collection. The vascular access device may include a pair of opposing pinch members configured to pinch the housing. The pair of opposing pinch members may be configured to move along the housing with the advancement element. The vascular access device may include a guidewire, which may include a wedge. In response to moving the advancement element distally along the housing, the pair of opposing pinch members may move the wedge distally and the guidewire may be advanced distally. The proximal end of the guidewire may include a looped portion, and in response to moving the advancement element distally along the housing, the pair of opposing pinch members may be aligned with an opening formed by the looped portion and advance the guidewire distally.

### CROSS-REFERENCE TO RELATED APPLICATION

The present application claims priority to U.S. Provisional Application Ser. No. 63/218,143, entitled “Vascular Access Device with Non-Contact Guidewire Advancement”, filed Jul. 2, 2021, the entire disclosure of which is hereby incorporated by reference in its entirety.

### BACKGROUND OF THE INVENTION

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter device includes a catheter that is over-the-needle. As its name implies, the catheter that is over-the-needle may be mounted over an introducer needle having a sharp distal tip. A catheter assembly may include a catheter hub, the catheter extending distally from the catheter hub, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

Infusion and blood withdrawal using the catheter may be difficult for several reasons, particularly when an indwelling time of the catheter increases. A fibrin sheath or thrombus may form on an internal surface of the catheter assembly, an external surface of the catheter assembly, or within the vasculature near or downstream of the distal tip of the catheter. The fibrin sheath or thrombus may block or narrow a fluid pathway through the catheter, which may impair infusion and/or collection of a high-quality blood sample.

The subject matter claimed herein is not limited to embodiments that solve any disadvantages or that operate only in environments such as those described above. Rather, this background is only provided to illustrate one example technology area where some implementations described herein may be practiced.

### SUMMARY OF THE INVENTION

The present disclosure relates generally to vascular access devices and related systems and methods. In some embodiments, a vascular access device may include an advancement element and a housing extending through the advancement element. In some embodiments, the housing may include a lumen extending therethrough, which may be used for blood collection. In some embodiments, the vascular access device may include a pair of opposing pinch members configured to pinch the housing, which may include an extension tube. In some embodiments, the pair of opposing pinch members may be disposed within the advancement element and configured to move along the housing with the advancement element.

In some embodiments, the vascular access device may include a guidewire, which may include a distal end and a proximal end. In some embodiments, the proximal end of the guidewire may include a wedge. In some embodiments, the guidewire may be disposed within the lumen. In some embodiments, in response to moving the advancement element distally along the housing, the pair of opposing pinch members may move the wedge distally and the guidewire may be advanced distally.

In some embodiments, the wedge may be monolithically formed as a single unit with the distal end of the guidewire. In some embodiments, the wedge may be cylindrical. In some embodiments, an outer surface of the wedge may include one or more grooves configured to allow fluid to pass the wedge. In some embodiments, the wedge may include one or more fins. In some embodiments, the wedge may be spherical or cylindrical. In some embodiments, the wedge may be constructed of metal or another suitable material.

In some embodiments, the pair of opposing pinch members may be disposed within the advancement element proximal to the wedge. In some embodiments, the vascular access device may include another pair of opposing pinch members configured to pinch the housing. In some embodiments, the other pair of opposing pinch members may be disposed within the advancement element distal to the wedge and configured to move along the housing with the advancement element, wherein in response to moving the advancement element proximally along the housing, the pair of opposing pinch members may move the wedge proximally and the guidewire may be retracted proximally. In some embodiments, an inner surface of the advancement element may include multiple bumps in contact with the pair of opposing pinch members, which may reduce friction.

In some embodiments, the vascular access device may include a distal adapter configured to couple to a catheter assembly. In some embodiments, a distal end of the housing may be coupled to the distal adapter. In some embodiments, the distal adapter may include a shaft and opposing lever locks. In some embodiments, the vascular access device may include a proximal connector coupled to a proximal end of the housing. In some embodiments, the proximal connector may be configured to couple to a blood collection device. In some embodiments, the guidewire may be coiled to form the wedge.

In some embodiments, the proximal end of the guidewire may include a looped portion formed by the guidewire. In some embodiments, the guidewire may be disposed within the lumen. In some embodiments, in response to moving the advancement element distally along the housing, the pair of opposing pinch members may be aligned with an opening formed by the looped portion and may advance the guidewire distally.

In some embodiments, the looped portion may include a single loop. In some embodiments, the single loop may be circular or ovular. In some embodiments, the looped portion may include multiple loops that may be stacked. In some embodiments, each of the loops may be circular or ovular. In some embodiments, the guidewire may include a twist portion proximate the looped portion. In some embodiments, a first end and a second end of the looped portion may be twisted together to form the twist portion.

In some embodiments, the loops may include one or more of a first loop, a second loop, and a third loop. In some embodiments, the loops may include the first loop and the second loop, and the first end of the looped portion may be proximate the first loop and the second end of the looped portion may be proximate the second loop. In some embodiments, the loops may include the first loop, the second loop, and the third loop, and the second loop may be in between the first loop and the third loop. In some embodiments, the first end of the looped portion may be proximate the first loop and the second end of the looped portion may be proximate the third loop. In some embodiments, the looped portion may include a coating.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

Example embodiments will be described and explained with additional specificity and detail through the use of the accompanying drawings in which:

FIG. 1A is an upper perspective view of an example vascular access device, illustrating an example advancement element in an example retracted position, according to some embodiments;

FIG. 1B is an upper perspective view of the vascular access device, illustrating the advancement element in an example advanced position, according to some embodiments;

FIG. 2A is a cross-sectional view of the vascular access device, according to some embodiments;

FIG. 2B is an upper perspective view of an example guidewire, according to some embodiments;

FIG. 2C is an example proximal end of the guidewire, according to some embodiments;

FIG. 2D is another example proximal end of the guidewire, according to some embodiments;

FIG. 2E is another example proximal end of the guidewire, according to some embodiments;

FIG. 2F is an upper perspective view of an example piece of the advancement element, according to some embodiments;

FIG. 2G is an upper perspective view of another example piece of the advancement element, configured to couple to the piece of FIG. 2F, according to some embodiments;

FIG. 3A is an upper perspective view of the guidewire, illustrating an example spherical proximal end, according to some embodiments;

FIG. 3B is a cross-sectional view of the advancement element, illustrating the spherical proximal end, according to some embodiments;

FIG. 4A is a top view of the guidewire, illustrating an example looped portion including an example single loop, according to some embodiments;

FIG. 4B is a side view of the guidewire, illustrating the looped portion including the single loop, according to some embodiments;

FIG. 4C is another top view of the guidewire, illustrating an example coating on the single loop, according to some embodiments;

FIG. 4D is a cross-sectional view of the advancement element, illustrating the looped portion including the single loop, according to some embodiments;

FIG. 5A is a side view of the guidewire, illustrating the looped portion including an example first loop and an example second loop, according to some embodiments;

FIG. 5B is a top view of the guidewire, illustrating the looped portion including the first loop, the second loop, and the coating, according to some embodiments;

FIG. 5C is a cross-sectional view of the advancement element, illustrating the looped portion including the first loop and the second loop, according to some embodiments;

FIG. 6A is a side view of the guidewire, illustrating the looped portion including the first loop, the second loop, and an example third loop, according to some embodiments;

FIG. 6B is a cross-sectional view of the advancement element, illustrating the looped portion including the first loop, the second loop, and the third loop, according to some embodiments;

FIG. 7 is an upper perspective view of the guidewire, illustrating an example coiled portion, according to some embodiments;

FIG. 8A is an upper perspective view of the vascular access device coupled to an example catheter assembly having a first configuration, according to some embodiments; and

FIG. 8B is an upper perspective view of the vascular access device coupled to the catheter assembly having a second configuration, according to some embodiments.

### DETAILED DESCRIPTION

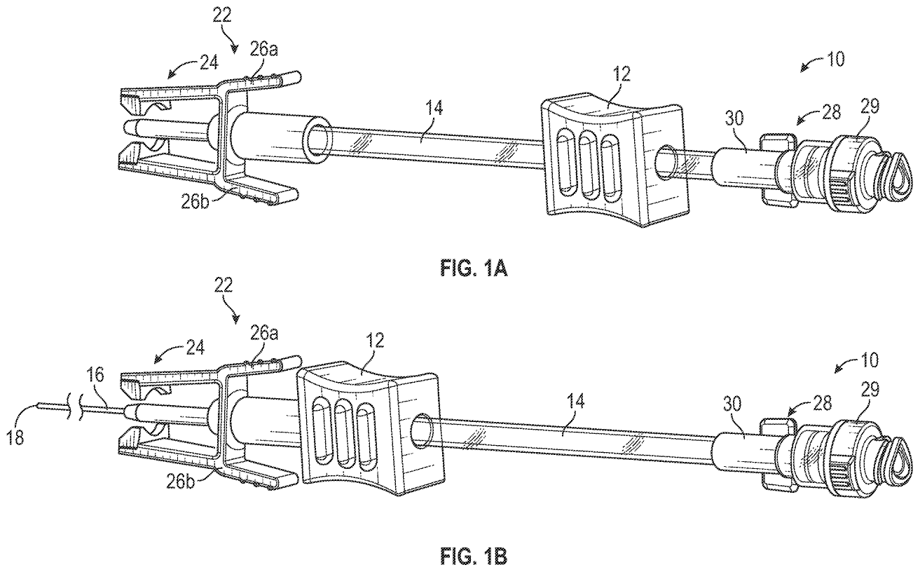

Referring now to FIGS. 1A-1B, a vascular access device 10 is illustrated, according to some embodiments. In some embodiments, the vascular access device 10 may include an advancement element 12 and a housing 14 extending through the advancement element 12. In some embodiments, the housing 14 may include an extension tube, which may be flexible or semi-flexible. In some embodiments, the housing 14 may include a coextruded guidewire to add stiffness to the housing 14. In some embodiments, the housing 14 may include a multi-lumen extension tube, which may be described further in U.S. patent application Ser. No. 17/574,127, filed Jan. 12, 2022, which is hereby incorporated by reference in its entirety.

In some embodiments, the vascular access device 10 may include a guidewire 16, which may be constructed of metal or another suitable material. In these and other embodiments, the guidewire may be lubricated or coated to ease advancement. Over time a catheter can become occluded at a catheter distal tip due to presence of fibrin sheath, thrombus, or vein walls or valves. In some embodiments, the guidewire 16 may be configured to extend into and/or through the catheter assembly to push through and/or disrupt an occlusion of the catheter when the vascular access device 10 is coupled to a catheter assembly that includes the catheter. In some embodiments, the guidewire 16 may be configured to advance distally through the catheter assembly and into the vasculature of the patient to overcome thrombus and fibrin sheath in or around the catheter assembly or in the vein that might otherwise prevent blood draw. In some embodiments, the guidewire 16 may be replaced by another suitable instrument, such as, for example, a tube. In some embodiments, the other suitable instrument may be similar or identical to the guidewire 16 in terms of one or more components and/or operation.

In some embodiments, the advancement element 12 may be configured to move between a retracted position illustrated, for example, in FIG. 1A, and an advanced position, illustrated, for example, in FIG. 1B. In some embodiments, the retracted position may correspond to an initial position of the advancement element 12. In some embodiments, the clinician may pinch or grasp the advancement element 12 to move the advancement element 12 between the retracted position and the advanced position. In some embodiments, in response to movement of the advancement element 12 distally from the retracted position to the advanced position, a distal end of the guidewire 16 may move from inside the housing 14 to outside of the housing 14.

In some embodiments, a distal end 22 of the vascular access device 10 may include a distal connector 24. In some embodiments, the distal connector 24 may include opposing lever arms 26a,26b. In some embodiments, distal ends of the opposing lever arms 26a,26b may be configured to move apart from each other in response to pressure applied to proximal ends of the opposing lever arms 26a,26b. In some embodiments, in response to removal of the pressure applied to the proximal ends of the opposing lever arms 26a,26b, the distal ends of the opposing lever arms 26a,26b may move closer to each other and clasp a portion of the catheter assembly, such as a needleless connector, another connector, or a proximal end of a catheter hub, for example. In some embodiments, the distal connector 24 may include a blunt cannula or male luer configured to insert into the portion of the catheter assembly.

In some embodiments, the distal connector 24 may include any suitable connector. For example, the distal connector 24 may include a threaded male luer, a slip male luer, a threaded male luer with a spin lock, a threaded male luer with a removable blunt cannula snap connection, a slip male luer with a removable blunt cannula snap connection, or another suitable connector. In some embodiments, the distal connector 24 may include one or more bond pockets, which may each be configured to receive an extension tube, which may be part of the catheter assembly or extend between the distal connector 24 and the catheter assembly. In some embodiments, the distal connector 24 may be monolithically formed as a single unit with the housing 14.

In some embodiments, the distal connector 24 may be configured to couple to the catheter assembly, which may be existing or already dwelling within the vasculature of the patient. In some embodiments, the catheter assembly may include a catheter adapter, which may include a distal end, a proximal end, and a lumen extending through the distal end of the catheter adapter and the proximal end of the catheter adapter.

In some embodiments, the catheter may extend from the distal end of the catheter adapter. In some embodiments, the catheter may include a peripheral intravenous catheter, a midline catheter, or a peripherally inserted central catheter. In some embodiments, the catheter assembly may include an introducer needle, which may extend through the catheter and facilitate piercing of skin and the vasculature to insert the catheter into the patient. In some embodiments, the introducer needle may be removed from the catheter assembly prior to coupling of the vascular access device 10 to the catheter assembly.

In some embodiments, the catheter assembly may be straight. In other embodiments, the catheter assembly may be integrated, having an extension tube that is integrated with the catheter adapter. Thus, a configuration may vary, according to some embodiments. In some embodiments, the catheter assembly may include an extension set, which may include the extension tube extending from and integrated with a side port of the catheter adapter. In some embodiments, the distal adapter 18 may be configured to couple to a portion of the catheter assembly, such as the proximal end of the catheter adapter and/or a needleless access connector. In some embodiments, the needleless access connector may be coupled to a proximal end, a T-connector, or another portion of the catheter assembly. In some embodiments, the needleless access connector may be permanently connected, such as, for example, via adhesive, to the distal adapter 18 to prevent intentional or unintentional removal by a user.

In some embodiments, a proximal end 28 of the vascular access device 10 may include a needleless connector 29 and/or a proximal connector 30, which may be similar to identical to the distal connector 24 or may include another suitable connector. In some embodiments, the proximal connector 30 may be configured to couple to a blood collection device. In some embodiments, the blood collection device may include a syringe, a BD VACUTAINER® one-use holder (available from Becton, Dickinson and Company of Franklin Lakes, N.J.), a BD VACUTAINER® LUER-LOK™ access device (also available from Becton, Dickinson and Company of Franklin Lakes, N.J.), or another suitable blood collection device, which may provide suction.

In some embodiments, the proximal connector 30 may be coupled to the blood collection device via the needleless connector 29, which may be disposed in between the blood collection device and the needleless connector 29. In some embodiments, the blood collection device and the proximal connector 30 may be monolithically formed as a single unit. In some embodiments, the proximal connector 30 may be monolithically formed as a single unit with the housing 14.

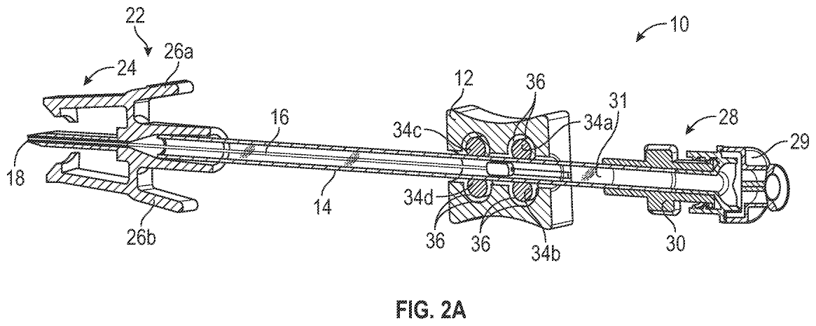

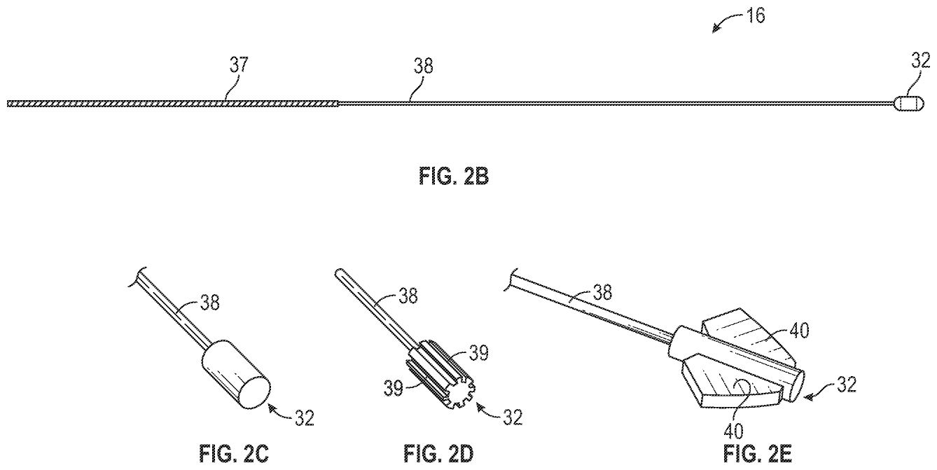

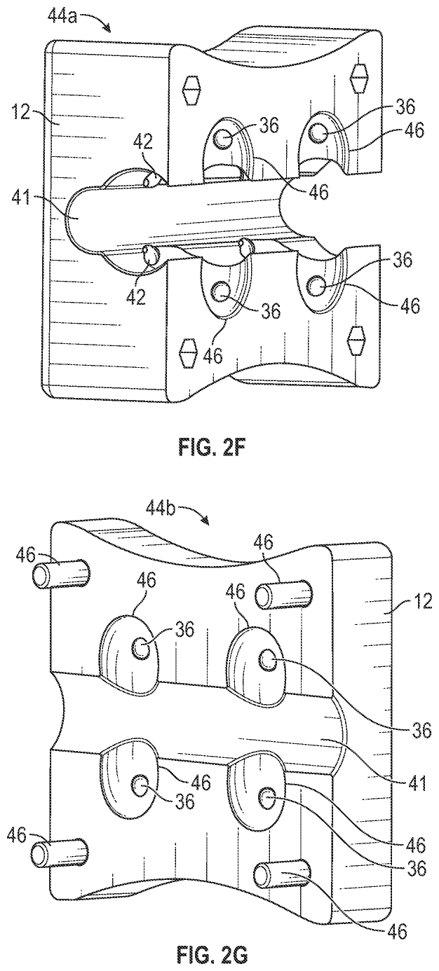

Referring now to FIG. 2A, in some embodiments, the housing 14 may include a lumen 31 extending through the housing 14. In some embodiments, a blood collection pathway through which blood flows during blood collection from the catheter assembly may include the distal connector 24, the lumen 31, and the proximal connector 30.

In some embodiments, the guidewire 16 may be moved to the advanced position prior to or during blood draw. In some embodiments, after completing a blood draw and before uncoupling the vascular access device 10 from the catheter assembly, the user may retract the guidewire 16 into the housing 14 by moving the advancement element towards the user or proximally. Thus, in some embodiments, a risk of exposure of the user to blood may be decreased.

In some embodiments, a proximal end of the guidewire 16 may include a wedge 32 disposed within the advancement element 12 and the lumen 31 of the housing 14. In some embodiments, the vascular access device 10 may include one or more pairs of opposing pinch members 34 configured to pinch the housing 14. In some embodiments, the pair of opposing pinch members 34a,b may be disposed within the advancement element 12 proximal to the wedge 32 and configured to move along the housing 14 with the advancement element. In some embodiments, in response to moving the advancement element distally along the housing 14, the pair of opposing pinch members 34a,b may compress the housing 14 to push the wedge 32 distally, and the guidewire 16 may be advanced distally.

In some embodiments, the vascular access device 10 may include another pair of opposing pinch members 34c,d configured to pinch the housing 14. In some embodiments, the other pair of opposing pinch members 34c,d may be disposed within the housing distal to the wedge 32 and configured to move along the housing 14 with the advancement element. In some embodiments, in response to moving the advancement element proximally along the housing 14, the pair of opposing pinch members 34c,d may compress the housing 14 and push the wedge 32 proximally and the guidewire 16 may be retracted proximally.

The pair of opposing pinch members 34a,b and the other pair of opposing pinch members 34c,d may be referred to collectively in the present disclosure as “opposing pinch members 34.” In some embodiments, in response to movement of the advancement element along the housing 14, the opposing pinch members 34 may rotate with respect to the advancement element and the housing 14. In some embodiments, in response to movement of the advancement element along the housing 14, the opposing pinch members 34 may rotate with respect to the advancement element and the housing 14. In some embodiments, an inner surface of the advancement element may include one or more bumps 36 in contact with the opposing pinch members 34, which may reduce friction as the opposing pinch members 34 rotate. In some embodiments, the wedge 32 and/or the opposing pinch members 34 may be lubricated with a lubricant, which may reduce friction.

In some embodiments, the opposing pinch members 34 may be constructed of plastic, metal, or another suitable material. In some embodiments, the opposing pinch members 34 may include spherical balls, ball bearings, wheels, or cylinders, which may be configured to rotate with respect to the advancement element. In some embodiments, the opposing pinch members 34 may include the wheels, which may be smooth or include feet along their edges. In these embodiments, lubricant may be applied to axels of the wheels to reduce friction. In some embodiments, the opposing pinch members 34 may be fixed with respect to the advancement element 12. For example, the opposing pinch members 34 may be molded into the advancement element 12.

In some embodiments, a number of the opposing pinch members 34 may vary based on a shape of the wedge 32. In some embodiments, the vascular access device 10 may include the pair of opposing pinch members 34a,b and the other pair of opposing pinch members 34c,d in response to the shape of the wedge 32 being cylindrical, for example. In some embodiments, the vascular access device 10 may include a single pair of the opposing pinch members 34, such as the pair of the opposing pinch members 34a,b, in response to the wedge 32 including a dog bone shape, and the single pair may be disposed in a middle or depression of the dog bone shape.

Referring now to FIG. 2B, in some embodiments, the distal end of the guidewire 16 may include a spring or coil 37, which may facilitate movement of a thrombus within the catheter assembly while also allowing fluid to flow the distal end of the guidewire 16. In some embodiments, the spring or coil 37 may include varying pitches along a length of the spring or coil. For example, a pitch of the spring or coil 37 upstream from or proximal to the catheter distal tip may be larger to facilitate more blood flow and increase flow rate, and a pitch of the spring or coil 37 configured to be positioned near the catheter distal tip may be smaller to prevent blood clots from entering the catheter distal tip, while still allowing blood to flow through the catheter distal tip. In some embodiments, the guidewire 16 may include a rod 38, which may extend through a center portion of the spring or coil 37. In some embodiments, the distal end of the guidewire 16 may include the rod 38 and may not include the spring or coil 37.

In some embodiments, the wedge 32 may be oval shaped, which may include a generally-oval shape. In some embodiments, rounded edges of the oval shape may facilitate an ability of the wedge 32 to round corners.

In some embodiments, the wedge 32 may be monolithically formed as a single unit with a distal end of the guidewire 16. For example, the proximal end of the guidewire 16 may be tapered or stepped outwardly such that an enlarged outer diameter forms the wedge 32. In other embodiments, the wedge 32 may be secured to the rod 38 with an adhesive, a weld, a press fit, a crimp, a wrapped wire, or other suitable mechanism. In some embodiments, the wedge 32 may be formed by melting the wedge 32 into a desired shape onto the guidewire 16.

In some embodiments, the distal end of the guidewire 16 may be constructed of metal, which may facilitate bending and/or flexibility to displace the thrombus. In some embodiments, the wedge 32 may be constructed of metal, plastic, or another suitable material. In some embodiments, the wedge 32 may include a bead, which may be constructed of metallic, plastic, or another suitable material. In some embodiments, the bead may be welded on the guidewire 16 to form the wedge 32. In some embodiments, the bead may be crimped on the guidewire 16 to form the wedge 32. In some embodiments, the bead may be secured to the guidewire 16 in another suitable manner.

Referring now to FIG. 2C, in some embodiments, the wedge 32 may be cylindrical, which may include a generally cylindrical shape. In some embodiments, opposing faces of the generally cylindrical shape of the wedge 32 may facilitate contact of the wedge 32 with the housing 14 when the housing 14 is pinched by the pair of opposing pinch members 34a,b and/or the other pair of opposing pinch members 34c,d.

Referring now to FIG. 2D, an outer surface of the wedge 32 may include one or more fenestrations or grooves 39 configured to allow fluid to pass through the wedge 32. Thus, in some embodiments, blood may flow proximally through the housing 14 and into the blood collection container. In some embodiments, the grooves 39 may be straight and aligned with a longitudinal axis of the housing 14, which may facilitate a reduced blood collection time. In these and other embodiments, the housing 14 may include a single lumen through which blood may flow. In some embodiments, the housing 14 may include the single lumen and blood may flow through or around the wedge 32. In some embodiments, the housing 14 may be multi-lumen, and blood may flow in a separate lumen from the wedge 32.

Referring now to FIG. 2E, in some embodiments, the wedge 32 may include one or more fins 40. In some embodiments, the fins 40 may facilitate fluid or blood to travel proximally through the housing 14 while also facilitating contact of the wedge 32 with the housing 14 when the housing 14 is pinched by the pair of opposing pinch members 34a,b and/or the other pair of opposing pinch members 34c,d.

Referring now to FIG. 2F-2G, in some embodiments, the advancement element may include an aperture 41 extending therethrough and configured to receive the housing 14. In some embodiments, the inner surface of the advancement element may include one or more protrusions 42, which may contact the housing 14 to reduce friction between the housing 14 and the advancement element 12 as the advancement element 12 moves along the housing 14. In some embodiments, the advancement element 12 may include multiple pieces 44a,b, which may be coupled together via one or more pegs 45 or any other suitable mechanism. In other embodiments, the advancement element 12 may be monolithically formed as a single unit.

In some embodiments, the advancement element 12 may include multiple cutouts 46, which may include the bumps 36. In these and other embodiments, the opposing pinch members 34 may include the spherical balls. In some embodiments, the cutouts 46 may be generally spherical and/or may extend outwardly from the aperture 41. In some embodiments, halves of the cutouts 46 illustrated in the multiple pieces 44a,b may be joined together to form the cutouts 46.

In some embodiments, the advancement element 12 may be rigid or semi-rigid to facilitate gripping and/or one-handed advancement by the user. In some embodiments, the advancement element 12 may include one or more grip features or a shape to facilitate gripping by the user. In some embodiments, the grip features may include one or more of ridges, indents, and tabs on a top of the advancement element 12 and/or one or more sides of the advancement element 12. In some embodiments, the shape of the advancement element 12 may include a square, cylinder, dog bone, or another suitable shape. In some embodiments, the shape of the advancement element 12 and/or the grip features may facilitate the user advancing and/or retracting the guidewire 16 without contacting the guidewire 16, thereby decreasing a risk of contamination and/or infection. In some embodiments, the advancement element 12 may include one or more textured surfaces to facilitate gripping by the user.

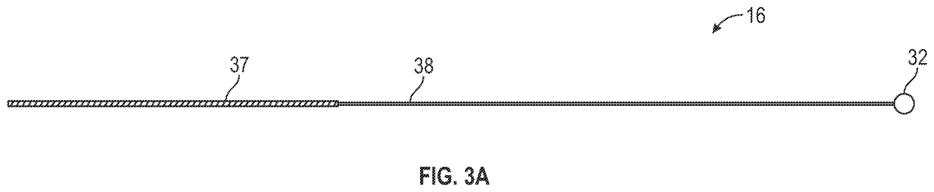

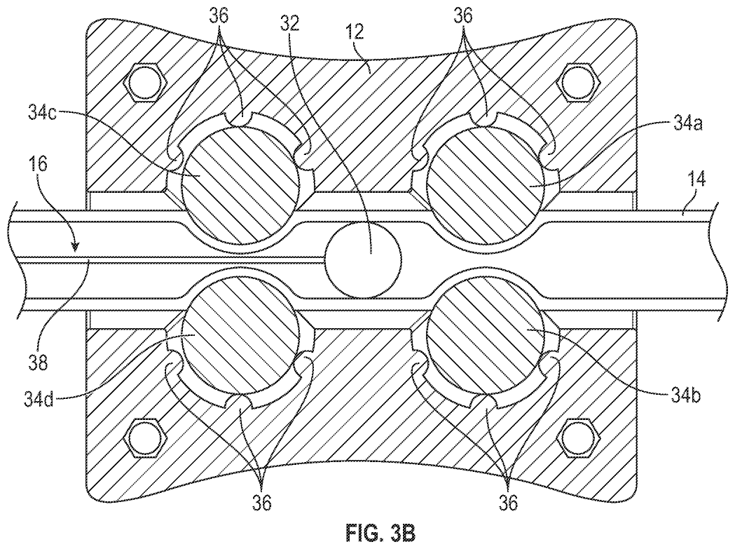

Referring now to FIGS. 3A-3B, in some embodiments, the wedge 32 may be spherical, which may include a generally-spherical shape. In some embodiments, the wedge 32 that is spherical may facilitate smooth movement of the wedge 32 within the lumen 31.

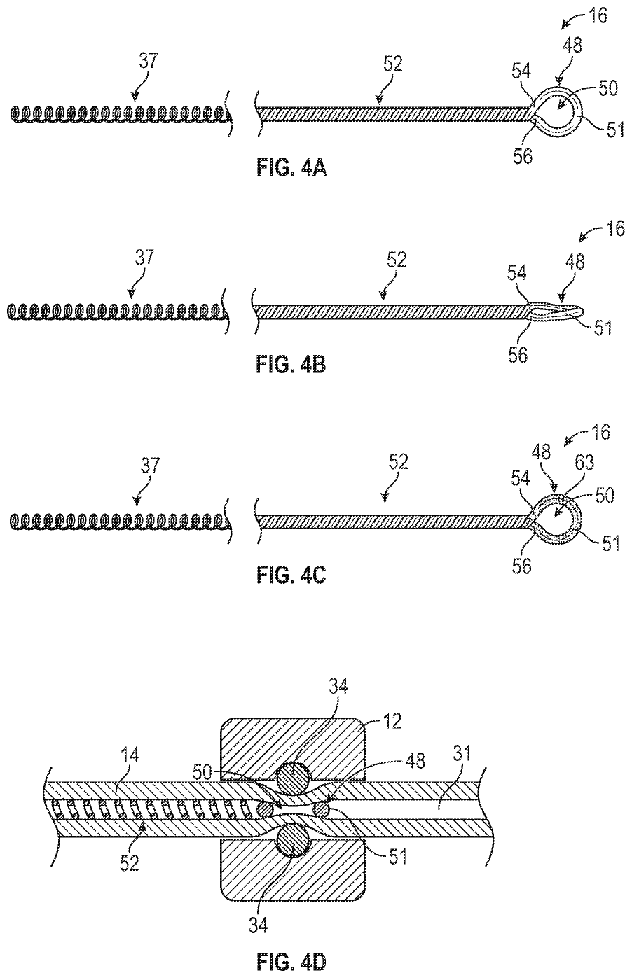

Referring now to FIGS. 4A-4D, in some embodiments, the proximal end of the guidewire 16 may include a looped portion 48 formed by the guidewire 16. In some embodiments, the guidewire 16 may be disposed within the lumen 31. In some embodiments, in response to moving the advancement element 12 distally along the housing 14, the pair of opposing pinch members 34 may be aligned with an opening 50 formed by the looped portion 48 and may advance the guidewire 16 distally. In some embodiments, the pair of opposing pinch members 34 may pinch or compress the housing 14 such that the housing 14 is disposed within the opening 50. In some embodiments, when the advancement element 12 moves along the housing 14, the housing 14 may stay within the opening 50.

In some embodiments, the looped portion 48 may include a single loop 51 or no more than one loop, which may be circular or ovular. In some embodiments, the guidewire 16 may include a twist portion 52, which may be proximate the looped portion 48. In some embodiments, a first end 54 and a second end 56 of the looped portion 48 may be twisted together to form the twist portion 52. In some embodiments, an entirety of the guidewire 16 may be monolithically formed as a single unit. In some embodiments, the guidewire 16 may be constructed with a single strand of metal or another suitable material.

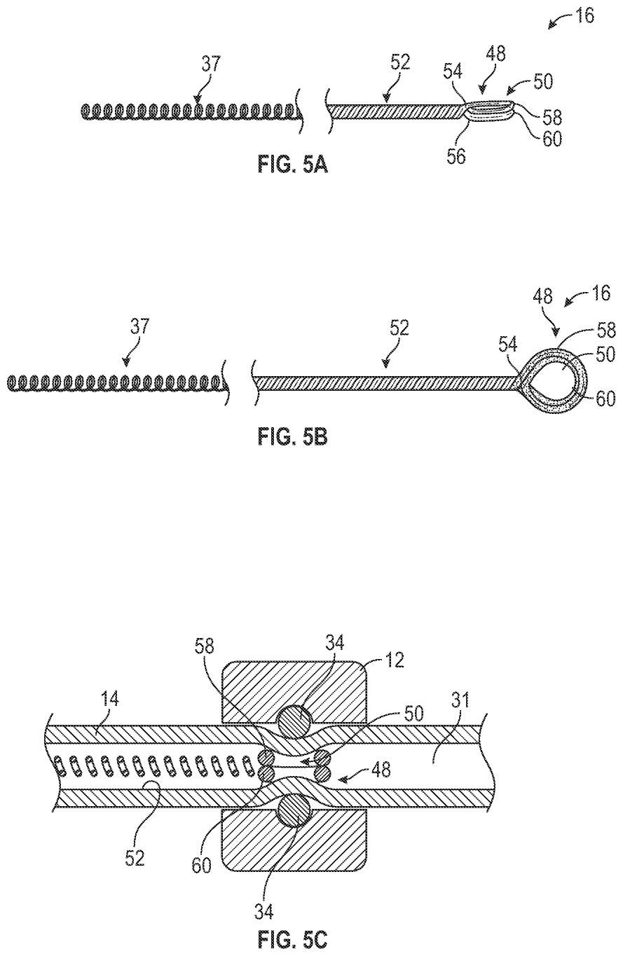

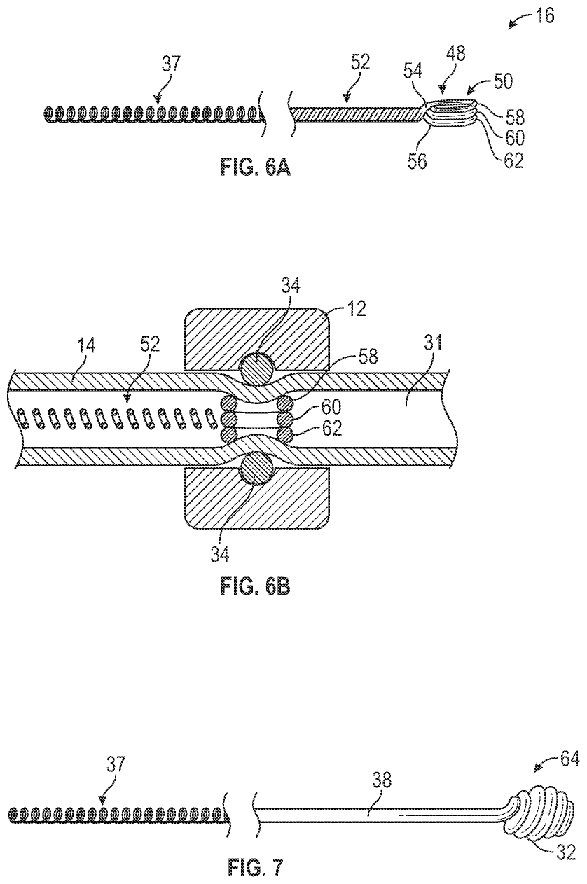

Referring now to FIGS. 5A-6B, in some embodiments, the looped portion 48 may include multiple loops that may be stacked on top of each other. In some embodiments, the guidewire 13 may be wrapped around an axis to form the loops. In some embodiments, the axis may be generally perpendicular to a longitudinal axis of the guidewire 13 and/or the longitudinal axis of the housing 14. In some embodiments, each of the loops may be circular or ovular, which may facilitate pinching by the pair of opposing pinch members 34. In some embodiments, the loops may include one or more of a first loop 58, a second loop 60, and a third loop 62. In some embodiments, the loops may include more than three loops. In some embodiments, the second loop 60 and/or the third loop 62 may increase a width of the proximal end of the guidewire 16 to be pinched by the pair of opposing pinch members 34, and thus may facilitate the lumen 31 being wider and configured for greater blood flow.

As illustrated in FIG. 4C, in some embodiments, the looped portion 48 may be coated with a coating 63, which may decrease friction between the looped portion 48 and the housing 14. In some embodiments, the coating 63 may include a lubricant.

As illustrated in FIGS. 5A-5C, in some embodiments, the loops may include the first loop 58 and the second loop 60, and the first end 54 of the looped portion 48 may be proximate the first loop 58 and the second end 56 of the looped portion 48 may be proximate the second loop 60. As illustrated in FIGS. 6A-6B, in some embodiments, the loops may include the first loop 58, the second loop 60, and the third loop 62, and the second loop 60 may be in between the first loop 58 and the third loop 62. In some embodiments, the first end 54 of the looped portion 48 may be proximate the first loop 58 and the second end 56 of the looped portion 48 may be proximate the third loop 62.

Referring now to FIG. 7, in some embodiments, to form the wedge 32, the proximal end of the guidewire 16 may include a coiled portion 64. In some embodiments, the coiled portion 64 may be coiled around an axis generally parallel or aligned with a longitudinal axis of the guidewire 16 and/or the rod 38. In some embodiments, each coil in the coiled portion 64 may contact an adjacent distal coil and/or an adjacent proximal coil of the coiled portion 64, such that the wedge 32 is compact. In some embodiments, the wedge 32 may be welded at one or more of the following locations to prevent stretching or separation of the coiled portion 64: a proximal end of the coiled portion 64, a distal end of the coiled portion 64, and between the proximal end of the coiled portion 64 and the distal end of the coiled portion 64.

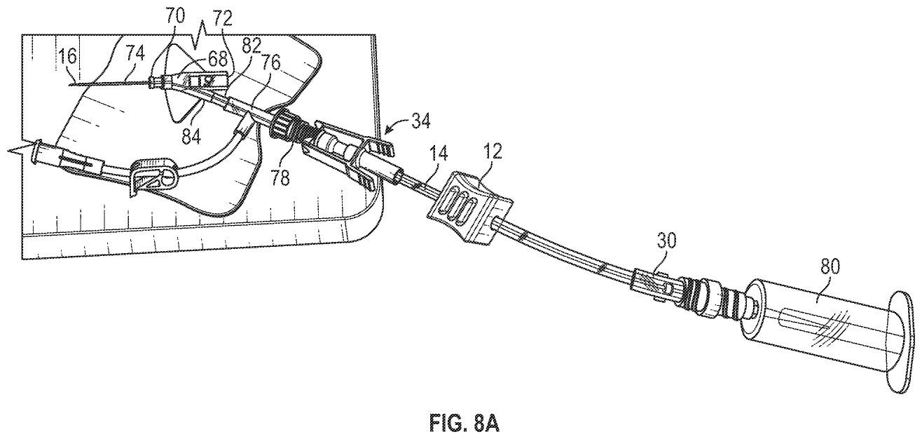

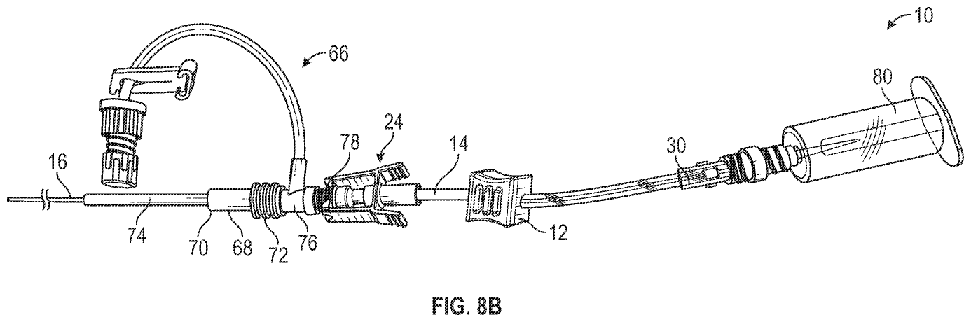

Referring now to FIGS. 8A-8B, in some embodiments, the distal connector 24 may be configured to couple to a catheter assembly 66, which may be existing or already dwelling within the vasculature of the patient. In some embodiments, the catheter assembly 66 may include a catheter adapter 68, which may include a distal end 70, a proximal end 72, and a lumen extending through the distal end 70 of the catheter adapter 68 and the proximal end 72 of the catheter adapter 68.

In some embodiments, a catheter 74 may extend from the distal end 70 of the catheter adapter 68. In some embodiments, the catheter 74 may include a peripheral intravenous catheter, a midline catheter, or a peripherally inserted central catheter. In some embodiments, the catheter assembly 66 may include an introducer needle (not illustrated), which may extend through the catheter 74 and facilitate piercing of skin and the vasculature to insert the catheter 74 into the patient. In some embodiments, the introducer needle may be removed from the catheter assembly prior to coupling of the vascular access device 10 to the catheter assembly 66.

In some embodiments, the catheter assembly 66 may be straight, as illustrated, for example, in FIG. 8B. In some embodiments, the catheter assembly 66 may include a T-connector 76 and/or a needleless access connector 78. In some embodiments, the distal connector 24 may be directly coupled to the T-connector 76 or needleless access connector 78. In some embodiments, during blood collection, blood may be configured to flow proximally through one or more of the following: the catheter 74, the catheter adapter 68, the T-connector 76, the needleless access connector 78, the distal connector 24, the housing 14, and the proximal connector 30.

In some embodiments, a blood collection device 80 may be coupled to the proximal connector 30 as mentioned previously. In some embodiments, the blood collection device 80 may include a syringe, a BD VACUTAINER® one-use holder (available from Becton, Dickinson and Company of Franklin Lakes, N.J.), a BD VACUTAINER® LUER-LOK™ access device (also available from Becton, Dickinson and Company of Franklin Lakes, N.J.), or another suitable blood collection device, which may provide suction. In response to movement of the advancement element 12 from the retracted position to the advanced position, the guidewire 16 may be configured to extend beyond the catheter distal tip.

As illustrated, for example, in FIG. 8A, in some embodiments, the catheter assembly 66 may be integrated, having an extension tube 82 that may be integrated with the catheter adapter 68. In some embodiments, the catheter assembly 66 may include an extension set, which may include the extension tube 82 extending from and integrated with a side port 84 of the catheter adapter 68. In some embodiments, the extension tube 82 may be coupled to the T-connector 76, which may be coupled to the needleless access connector 78.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed is:

1. A vascular access device, comprising:
an advancement element;
a housing extending through the advancement element, wherein the housing comprises a lumen extending therethrough;
a pair of opposing pinch members configured to pinch the housing, wherein the pair of opposing pinch members are disposed within the advancement element and configured to move along the housing with the advancement element; and
an instrument comprising a distal end and a proximal end, wherein the proximal end of the instrument comprises a wedge, wherein the instrument is disposed within the lumen, wherein in response to moving the advancement element distally along the housing, the pair of opposing pinch members move the wedge distally and the instrument is advanced distally.

2. The vascular access device of claim 1, wherein the wedge is monolithically formed as a single unit with the distal end of the instrument.

3. The vascular access device of claim 1, wherein the wedge is cylindrical.

4. The vascular access device of claim 1, wherein an outer surface of the wedge comprises a plurality of grooves configured to allow fluid to pass the wedge.

5. The vascular access device of claim 1, wherein the wedge comprises a plurality of fins.

6. The vascular access device of claim 1, wherein the wedge is spherical.

7. The vascular access device of claim 1, wherein the wedge is constructed of metal.

8. The vascular access device of claim 1, wherein the pair of opposing pinch members are disposed within the advancement element proximal to the wedge, further comprising another pair of opposing pinch members configured to pinch the housing, wherein the other pair of opposing pinch members are disposed within the advancement element distal to the wedge and configured to move along the housing with the advancement element, wherein in response to moving the advancement element proximally along the housing, the pair of opposing pinch members move the wedge proximally and the instrument is retracted proximally.

9. The vascular access device of claim 1, wherein an inner surface of the advancement element comprises a plurality of bumps in contact with the pair of opposing pinch members.

10. The vascular access device of claim 1, further comprising a distal adapter configured to couple to a catheter assembly, wherein a distal end of the housing is coupled to the distal adapter.

11. The vascular access device of claim 10, wherein the distal adapter comprises a shaft and opposing lever locks.

12. The vascular access device of claim 1, further comprising a proximal connector coupled to a proximal end of the housing, wherein the proximal connector is configured to couple to a blood collection device.

13. The vascular access device of claim 1, wherein the instrument is coiled to form the wedge.

14. A vascular access device, comprising:
an advancement element;
a housing extending through the advancement element, wherein the housing comprises a lumen extending therethrough;
a pair of opposing pinch members configured to pinch the housing, wherein the pair of opposing pinch members are disposed within the advancement element and configured to move along the extension tube with the housing; and
a guidewire comprising a distal end and a proximal end, wherein the proximal end of the guidewire comprises a looped portion formed by the guidewire, wherein the guidewire is disposed within the lumen, wherein in response to moving the advancement element distally along the housing, the pair of opposing pinch members are aligned with an opening formed by the looped portion and advance the guidewire distally.

15. The vascular access device of claim 14, wherein the looped portion comprises a single loop, wherein the single loop is circular or ovular.

16. The vascular access device of claim 14, wherein the looped portion comprises a plurality of loops that are stacked, wherein each of the plurality of loops is circular or ovular.

17. The vascular access device of claim 16, wherein the guidewire further comprises a twist portion proximate the looped portion, wherein a first end and a second end of the looped portion are twisted together to form the twist portion.

18. The vascular access device of claim 17, wherein the plurality of loops comprises a first loop and a second loop, wherein the first end is proximate the first loop and the second end is proximate the second loop.

19. The vascular access device of claim 17, wherein the plurality of loops comprises a first loop, a second loop, and a third loop, wherein the second loop is in between the first loop and the third loop, wherein the first end is proximate the first loop and the second end is proximate the third loop.

20. The vascular access device of claim 14, wherein the looped portion comprises a coating or lubricant.
